(19) United States
(12) Patent Application Publication
(10) Pub. No.: US 2007/0202179 A1
(43) Pub. Date: Aug. 30, 2007

(54) Low Dose Pharmaceutical Products

(76) Inventors: Patrick Faulkner, King of Prussia, PA (US); Renna Pan, King of Prussia, PA (US); Gerard Provot, Evreux (FR)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/599,796

(22) PCT Filed: Apr. 7, 2005
(86) PCT No.: PCT/EP05/03722

§ 371 Date: Oct. 10, 2006

Related U.S. Application Data
(60) Provisional application No. 60/561,235, filed on Apr. 9, 2004.

(51) Int. Cl. A61K 9/14

(52) U.S. Cl. 424/489

(57) ABSTRACT

This invention relates to a method of formulating dosage forms of pharmaceutically active ingredients and to solid dosage forms produced thereby wherein the dosage form contains a very low dose, more especially an ultra-low dose of the pharmaceutically active ingredients.

### FIELD OF THE INVENTION

This invention relates to a method of formulating dosage forms of pharmaceutically active ingredients and to solid dosage forms produced thereby wherein the dosage form contains a very low dose, more especially an ultra-low dose of the pharmaceutically active ingredients. In particular, the invention provides methods and compositions comprising a very low dose of the pharmaceutically active ingredient 2-methyl-2-[4-{[(4-methyl-2-[4-trifluoromethylphenyl]-thiazol-5-ylcarbonyl)amino]methyl}phenoxy]propionic acid (Compound 1) or pharmaceutically acceptable salts, solvates and physiologically functional derivatives thereof. The compositions comprising Compound 1 are particularly useful for the prevention or treatment of PPAR mediated diseases or conditions.

### BACKGROUND TO THE INVENTION

It is recognised in the pharmaceutical field that, when formulating highly active pharmaceutically active ingredients for administration to those in need of therapy, the challenge is to ensure an even distribution of the pharmaceutically active ingredients throughout the pharmaceutical excipients to ensure a proper dosage and ensure homogeneity. This is particularly problematic with formulating very low-dose drugs—for example less than 100 μg. In such a scenario the technical problem is to ensure that the pharmaceutically active substance is distributed evenly among a comparatively large amount of excipient particles.

The simplest way of manufacturing tablets is simply to blend all the ingredients as dry powders and tablet them (“direct compression”). This is rarely successful for low-dose drugs; a common problem being segregation of the powder blend during tabletting. A variation of this method which has been successful for low dose drugs is known as “trituration”, and is sometimes referred to as “ordered mixing” or “interactive mixing”. Very fine particles of the drug are first mixed with a small portion of excipient; the product then mixed with a slightly larger portion of excipient and so on until the desired mix is obtained. This method relies on the fine drug particles adhering electrostatically to the larger excipient ones, thus preventing segregation. The method works with some drugs, but success depends on the surface properties of both drug and excipient, and the method is very laborious.

A preferred alternative method for formulating low dose drugs is known as “wet granulation”. The drug is dissolved in water or another solvent, and blended with solid excipients including a binder, for example povidone, to form a wet mass containing 5-20% by weight of solution to total weight of granulation mix, which is then dried off in a separate step. The binder causes particles of excipient to clump together, and as the mass dries these clumps (“granules”) either contain or are coated with the drug. WO 96/09056 describes a method of using wet granulation process to formulate low dose pharmaceutical dosage units. Dosage forms containing drugs in an amount of 0.005 to 1.0% by weight are formulated. However, there is no mention of content uniformity of the dosage forms. EP 0955048 A1 describes a process for preparation of pharmaceutical dosage units containing an active substance of from 0.005 to 1.0% by weight of micronised active pharmaceutical ingredient. Desired content uniformity (<3% RSD—(Relative Standard Deviation)) was achieved for drug content of greater than 0.005% by weight.

Fluid bed granulation has been used to achieve content uniformity of low dose (1 g-mg) tablets (Thiel et al., J. Pharm. Pharmacol. 1986, 38, 335-343). In this process, the micronised drug is blended as a powder with other excipients, then loaded into a fluid bed granulator, and the powders are agglomerated by spraying on a solution of a binder; drying takes place concomitantly. Tablets compressed from a granulation containing 0.001-2% active pharmaceutical ingredient had a content CV (Coefficient of Variance) of <5%. Although it met the specification of USP, the content uniformity is outside the desired limit of <3% RSD.

Another process for formulating low dose drugs is known as carrier granulation (Michoel et al., Pharmaceutical Technology June 1988, 66-84). This functions by spraying a solution of binder such as povidone in water onto relatively large excipient particles such as hydrous lactose and then spraying small dry drug substance particles onto that, thus coating the excipient with drug particles which are stuck on by the binder. The quantity of solution used was 3.3-3.5% by weight of solution to total granulation mix. The method was applied to a formulation containing 4-5% drug by weight. This method also requires drying; the drug particle size needs to be very small, which often requires an extra milling step and the very fine drug powder may not flow at all well.

Dahl et al., Drug Development and Industrial Pharmacy 1990, 16 (12),1881-1891, describes the preparation of solid capsule formulations using a spray-on liquid drug carrier. The model drug is dissolved in a non-volatile solvent, propylene carbonate, and sprayed onto a compressible sugar at a loading of around 0.01% by weight of drug to total solid, to give a final unit dose of 35 μg. The solvent, being non-volatile, remains in the blend. It is added at around 5% by weight of the total formulation; lower ratios of solvent to solid resulted in decreased ability to disintegrate and dissolve. The resulting, somewhat sticky, powder showed some difficulties in automated encapsulation machines, and would be likely to give significant problems in tabletting.

Yalkowski (U.S. Pat. No. 4,489,026) describes a process which involves very slowly spraying a dilute solution of drug in a volatile inert solvent, preferably an organic solvent having a boiling point lower than 80° C., onto excipient powder in an open coating pan; a continuous flow of air dries the product during the spraying process. This process was applied to drugs with a unit dose of 10 μg or less. The spray rate is limited to 1-10 ml/min, making the process suitable only for very small batch-sizes (the example quoted prepared 1000 tablets). The weight ratio of solution to carrier used was 15%; also, the use of volatile organic liquids is now regarded as a significant hazard, requiring solvent-recovery processes and explosion-proof equipment.

Katdare (U.S. Pat. No. 4,898,736) describes a simplified version of this process, suitable for unit doses of 50-1000 μg; the drug, dissolved in an easily evaporated solvent such as, ethanol, methanol, acetone or tetrahydrofuran, is simply blended with excipients in a ratio of 2.26% or 6.75% and then dried, followed by lubrication and tabletting. This process is in principle suitable for commercial scale manufacture, but does still have the problems associated with the use of volatile organic solvents.

WO 97/04750 describes the formulation of low-dose drugs comprising admixing carrier particles to a solution of drug in water in a quantity of 1-3% by weight of solution to total mix. Preferably the mixing step is carried out in a high shear mixer. During mixing, the carrier particles are coated with a thin film of drug substance. This process does not include the use of binders and disintegrants. Dosage units containing 5-125 μg were formulated. However no content uniformity data was disclosed.

It is an object of the present invention to provide further methods of preparing low dosage formulations (less than 100 μg), more particularly for preparing ultra-low dose formulations of pharmaceutically active ingredients (less than 1 μg dosages). In particular it is also preferable to provide methods which avoid the use of organic solvents.

### SUMMARY OF THE INVENTION

The present invention provides a method for preparing dosage forms comprising low dose pharmaceutically active substances which comprises admixing carrier particles with a solution comprising the pharmaceutically active substance together with a binder therefor. The resulting mixture may be formulated into suitable unit dose presentations, e.g. by tabletting and optionally film coating.

In a further aspect the present invention provides a pharmaceutical composition comprising 1-100 micrograms of 2-methyl-2-[4-{[(4-methyl-2-[4-trifluoromethyl phenyl]-thiazol-5-ylcarbonyl)amino]methyl}phenoxy]propionic acid or pharmaceutically acceptable salts, solvates and physiologically functional derivatives thereof together with a carrier therefor. The pharmaceutical composition may be prepared by the above method.

### DETAILED DESCRIPTION OF THE INVENTION

Any pharmaceutically active ingredient (drug substance) having a low effective dose and having a sufficient degree of solubility in the chosen solvent, preferably an aqueous solvent (water or aqueous buffer) may be formulated by the process of the invention. The concentration of drug in the solution is dependent on the unit dose of the drug required.

The optimum quantity of solution will depend on the absorbent qualities of the carrier particles, the stability of the drug and the characteristics of the mixing device. Too high a level of moisture is not desired because it would increase the cycle time for drying and add to the manufacturing cost, too low a level of moisture may impact homogeneity of granulation. The preferred ratio (w/w) of solution comprising drug and binder: carrier is 5-50:100, more preferably 15-35:100, even more preferably 20-30:100.

The process of the invention is particularly suitable for the preparation of dosage forms containing low doses of pharmaceutically active ingredients, particularly less than 100 μg of drug, more particularly less than 20 μg and most particularly less than 1 μg.

In an alternative embodiment, dosage forms wherein the drug substance may be less than or equal to 0.0001% w/w of the solid dosage form may be prepared by the process of the invention.

In particular, the process of the present invention is particularly useful for the preparation of dosage forms having content uniformity for drug content of <7.5%, preferably <6%, more preferably less than 3% RSD. In particular the dosage form is a solid dosage form.

The mixing step is preferably carried out in a High Shear Mixer (sometimes referred to as High Shear Granulator). During mixing the carrier particles will be evenly coated with a thin film of drug/binder solution. Some of the water naturally dries off during the mixing. If necessary a further drying stage can be carried out.

Suitable binders include starch, gelatin, natural sugars such as glucose or beta-lactose, corn sweeteners, natural and synthetic gums such as acacia, tragacanth or sodium alginate, carboxymethylcellulose, polyethylene glycol, povidone, waxes, hydroxypropylmethylcellulose (HPMC), hydroxypropylcellulose (HPC), polyvinylalcohol (PVA) and the like including any combination of suitable binders.

The carrier may comprise suitable pharmaceutical excipient or excipients well known in the art. The carrier(s) must be acceptable in the sense of being compatible with the other ingredients of the formulation and not deleterious to the recipient thereof.

Thus for example the carrier may be any suitable soluble, pharmaceutically acceptable excipient such as anhydrous lactose, lactose monohydrate, mannitol, or an insoluble, pharmaceutically acceptable excipient such as microcrystalline cellulose or dicalcium phosphate and the like including any combinations of carriers.

The carrier may include further pharmaceutical additives including but not limited to lubricants, fillers, disintegrants, colouring agents and flavouring agents as required and may be included before or after the carrier is admixed with the drug/binder solution.

Glidants and lubricants include as colloidal silica, talc, magnesium stearate, calcium stearate or solid polyethylene glycol. A disintegrating or solubilizing agent might be agar-agar, calcium carbonate or sodium carbonate.

Disintegrants include, without limitation, starch, sodium starch glycolate, crospovidone, croscarmellose, methyl-cellulose, agar, bentonite, xanthan gum and the like.

Further excipients which improve the chemical stability of the drug may also be included, such as acidic or alkaline excipients.

The resultant mixture may then be formulated into suitable finished forms. In a preferred aspect tablets are produced but other product forms may similarly be prepared by art methods such as capsules, suspensions, lozenges and will be apparent to a person skilled in the art and discussed in greater detail below.

The present invention further provides a pharmaceutical composition formulated in accordance with the process of the invention comprising a drug and the use of said composition as an active therapeutic substance.

The invention further provides a pharmaceutical composition obtainable in accordance with the process of the invention comprising a drug, and the use of said composition as an active therapeutic substance.

Pharmaceutical formulations may be presented in unit dose forms containing a predetermined amount of active ingredient per unit dose. Preferred unit dosage formulations are those containing a daily dose or sub-dose, or an appropriate fraction thereof, of an active ingredient. They may contain the active ingredient in the form of a salt, solvate or physiologically functional derivative thereof.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Capsules are made by preparing a mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, other ingredients including suitable lubricants, disintegrating agents and coloring agents can also be incorporated into the mixture as discussed above. Tablets may be formulated, for example, by preparing a powder mixture, granulating or slugging, adding a lubricant and disintegrant and pressing into tablets. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups, suspensions and elixirs can be prepared in dosage unit form so that a given quantity contains a predetermined amount of the compound.

Where appropriate, dosage unit formulations for oral administration can be microencapsulated. The formulation can also be prepared to prolong or sustain the release as for example by coating or embedding particulate material in polymers, wax or the like.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose.

Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

As used herein, the term “physiologically functional derivative” refers to any pharmaceutically acceptable derivative for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

The present invention also covers the use of salts of the pharmaceutically active compounds in the low and ultra low dose formulations, Typically, the salts of the present invention are pharmaceutically acceptable salts. Salts encompassed within the term “pharmaceutically acceptable salts” refer to non-toxic salts of the compounds of this invention. A pharmaceutically acceptable acid addition salt can be formed by reaction of a pharmaceutically active compound with a suitable inorganic or organic acid (such as hydrobromic, hydrochloric, sulfuric, nitric, phosphoric, succinic, maleic, formic, acetic, propionic, fumaric, citric, tartaric, lacetic, benzoic, salicylic, glutamaic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated for example by crystallisation and filtration. A pharmaceutically acceptable acid addition salt of a pharmaceutically active compound can comprise or be for example a hydrobromide, hydrochloride, sulfate, nitrate, phosphate, succinate, maleate, formate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate) or hexanoate salt.

A pharmaceutically acceptable base addition salt can be formed by reaction of a pharmaceutically active compound with a suitable inorganic or organic base (e.g. triethylamine, ethanolamine, triethanolamine, choline, arginine, lysine or histidine), optionally in a suitable solvent such as an organic solvent, to give the base addition salt which is usually isolated for example by crystallisation and filtration.

Other suitable pharmaceutically acceptable salts include pharmaceutically acceptable metal salts, for example pharmaceutically acceptable alkali-metal or alkaline-earth-metal salts such as sodium, potassium, calcium or magnesium salts; in particular pharmaceutically acceptable metal salts of one or more carboxylic acid moieties that may be present in the a pharmaceutically active compound

Other non-pharmaceutically acceptable salts, eg. oxalates, may be used, for example in the isolation of compounds of the invention, and are included within the scope of this invention.

The invention includes within its scope all possible stoichiometric and non-stoichiometric forms of the salts of the pharmaceutically active compounds. Other salts, which are not pharmaceutically acceptable, may be useful in the preparation of compounds of this invention and these form a further aspect of the invention.

The process of the invention is particularly suitable for the formulation of 2-methyl-2-[4-{[(4-methyl-2-[4-trifluoromethyl phenyl]-thiazol-5-ylcarbonyl)amino]methyl}phenoxy]propionic acid or pharmaceutically acceptable salts, solvates and physiologically functional derivatives thereof.

WO 01/40207 discloses certain compounds disclosed as having activity at human Peroxisome Proliferator Activated Receptor alpha (PPAR alpha). In particular, WO 01/40207 discloses the compound 2-methyl-2-[4-{[(4-methyl-2-[4-trifluoromethyl phenyl]-thiazol-5-ylcarbonyl)amino]methyl}phenoxy]propionic acid or pharmaceutically acceptable salts, solvates and physiologically functional derivatives thereof which may be represented by formula (1), hereinafter referred to as Compound 1.

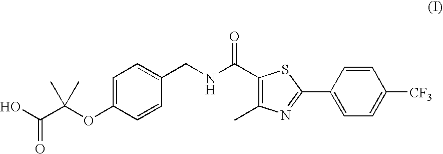

The compound of formula (1) is a particularly preferred PPAR alpha agonist and is described in WO 01/40207 as being of use in human PPAR alpha mediated diseases. The dosage regime contemplated in WO 01/40207 is 0.02-5000 mg per day. This compound is being investigated for dyslipidemia, syndrome X and atherosclerosis and surprisingly it has been found that the compound is effective at very low dosage regimes, particularly less than 0.02 mg per day. Thus the invention further provides a pharmaceutical composition comprising less than 1-100 μg Compound (1) or pharmaceutically acceptable salts, solvates and physiologically functional derivatives thereof together with a pharmaceutically acceptable carrier. Preferably the composition comprises less than 20 μg, more preferably 1-18 μg, most preferably 1-10 μg.

The carrier(s), diluent(s) or excipient(s) must be acceptable in the sense of being compatible with the other ingredients of the formulation and not deleterious to the recipient thereof.

The present invention therefore provides a method of treatment of a human PPAR alpha mediated disease or condition which comprises administration to a subject a daily dosage of 1-100 μg of a compound of formula (1) or pharmaceutically acceptable salts, solvates and physiologically functional derivatives thereof. Preferably the daily dosage is less than 20 μg, more preferably 1-18 μg, most preferably 1-10 μg.

There is further provided the use of a compound of formula (1) or pharmaceutically acceptable salts, solvates and physiologically functional derivatives thereof in a daily dose of 1-100 μg in the manufacture of a medicament for the treatment of a hPPAR mediated disease or condition. Preferably the daily dosage is less than 20 μg, more preferably 1-18 μg, most preferably 1-10 μg.

Human (h) PPAR mediated diseases or conditions include dyslipidemia including associated diabetic dyslipidemia and mixed dyslipidemia, syndrome X (as defined in this application this embraces metabolic syndrome), heart failure, hypercholesteremia, cardiovascular disease including atherosclerosis, arteriosclerosis, and hypertriglyceridemia, type II diabetes mellitus, type I diabetes, insulin resistance, hyperlipidemia, inflammation, epithelial hyperproliferative diseases including eczema and psoriasis and conditions associated with the lung and gut and regulation of appetite and food intake in subjects suffering from disorders such as obesity, anorexia bulimia, and anorexia nervosa, cancer, Alzheimers disease or other cognitive disorders.

Preferred human PPAR mediated diseases or conditions are atherosclerosis, syndrome X and dyslipidemia.

The compound of formula (1) may be prepared, e.g., by the methods described in WO 01/40207.

WO 02/096893 describes a particular route of synthesis of the compound of formula (1), together with the identification of particular polymorphic forms. These preferred forms are identified as form 2 and form 6. Thus preferably the compound of formula (1) comprises form 2, form 6 and mixtures thereof.

Pharmaceutical formulations of Compound (1) may be presented in unit dose forms containing a predetermined amount of active ingredient per unit dose. Suitable unit doses to achieve such daily doses include 10 μg administered once daily, 5 μg administered twice daily. Preferred unit dosage formulations are those containing a daily dose or sub-dose, as herein above recited, or an appropriate fraction thereof, of an active ingredient.

Pharmaceutical formulations of Compound (1) may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s), as discussed above.

In a particular aspect, preferably the low dose formulations of Compound (1) are prepared by the process of the present invention. Preferably the binder is Povidone.

Thus the present invention provides a pharmaceutical composition formulated in accordance with the process of the invention comprising 1-100 μg Compound (1) or pharmaceutically acceptable salts, solvates and physiologically functional derivatives thereof and the use of said composition as an active therapeutic substance, in particular in the treatment of hPPAR mediated diseases or disorders. Preferably the composition comprises less than 20 μg, more preferably 1-18 μg, most preferably 1-10 μg of Compound (1) or pharmaceutically acceptable salts, solvates and physiologically functional derivatives thereof.

The invention is further illustrated by the following examples which should not be construed as constituting a limitation thereto.

### EXAMPLE 1

### Formulation of Tablets Containing 10 μg Compound (1) by Spray Coating

Compound 1 drug substance and povidone were dissolved in ethanol to make a spray solution. The solution contained 0.06% Compound 1 and 15% povidone. The solution was then spray coated onto Lactose DT in a Wurster coater to produce a granulation. About 200 g of solution was coated onto 1 kg of Lactose DT. The resultant granulation which contained 0.012% Compound 1 was blended with Avicel PH102 and the blend was lubricated using magnesium stearate. The lubricated blend was compressed into 6 mm round tablets, with a targeted tablet weight of 120 mg and hardness of 60 kN.

TABLE 1
Formulation of Tablets containing 10 μg Compound 1

| | |
|---|---|
| | Unit Formula |
| Ingredients | (mg/tablet) |

| | | |
|---|---|---|
| Compound 1 Granulation: | 84.0 | |
| Compound 1 drug substance | | 0.010 |
| Povidone (Plasdone K29/32) | | 2.52 |
| Ethanol* | | 14.3 |
| Lactose Monohydrtate (Lactose DT) | | 81.5 |
| Microcrystalline Cellulose (Avicel PH 102) | 34.8 | |
| Magnesium Stearate | 1.20 | |
| Total | 120.0 | |

### EXAMPLE 2

### Formulation of Tablets Containing 10 μg Compound (1) by Fluid Bed Coating

A fluid bed coating process was used to manufacture Compound 110 μg tablets. The formula for this process example is presented in Table 2.

TABLE 2
Formulation of Tablets containing 10 μg
Compound 1 by Fluid Bed Coating Process

| | Unit Formula | |
|---|---|---|
| Ingredients | (mg/tablet) | |
| Compound 1 Granulation | 84.0 | |
| Compound 1 drug substance | | 0.010 |
| Povidone (Plasdone K29/32) | | 2.44 |
| Water* | | 14.3 |
| Sodium Hydroxide** | | QS |
| Lactose Monohydrate (Pharmatose DCL15) | | 81.5 |
| Microcrystalline Cellulose (Avicel PH 102) | 34.8 | |
| Magnesium Stearate | 1.20 | |
| Total | 120.0 | |

*Water was removed during the drying process
**A 1.0N sodium hydroxide solution was prepared for pH adjustment

Povidone (PVP) was dissolved in water and the PVP solution was adjusted to a pH of 7-10 using 1.0N NaOH. Compound 1 was then dissolved in the PVP solution. The resulting solution contained 0.06% Compound 1 and 15% povidone.

The Compound 1 solution was spray coated onto Pharmatose DCL15 in a Wurster coater to produce a Compound 1 granulation. About 200 g of Compound 1 solution was coated onto 1 kg of Pharmatose DCL15. The Compound 1 granulation which contained 0.012% Compound 1 was blended with Avicel PH102 and the blend lubricated using magnesium stearate. The lubricated Compound 1 blend was compressed into ¼″ round tablets. The tablet weight is 120 mg, the target hardness is 6-7 kP.

The Compound 110 μg tablets were tested for content uniformity. The test results are presented in Table 3.

TABLE 3
Content Uniformity of Tablets containing 10 μg Compound
1 Manufactured by Fluid Bed Coating Process

| Sample | mg/tablet |
|---|---|
| 1 | 9.92 |
| 2 | 10.12 |
| 3 | 10.14 |
| 4 | 9.88 |
| 5 | 9.70 |
| 6 | 10.59 |
| 7 | 9.86 |
| 8 | 9.80 |
| 9 | 9.94 |
| 10 | 9.90 |
| Average | 9.99 |
| % RSD | 2.5 |
| Maximum | 10.59 |
| Minimum | 9.70 |

The data in Table 3 indicates that the preferred content uniformity (<3% RSD) was achieved by using the Fluid bed coating process.

### EXAMPLE 3

### Formulation of 2-methyl-2-[4-{[(4-methyl-2-[4-trifluoromethyl phenyl]-thiazol-5-ylcarbonyl)amino]methyl}phenoxy]propionic acid (Compound (1) 10 μg Tablets by High Shear Granulation Process

Sodium phosphate monobasic and povidone were dissolved in water and the solution was adjusted to pH of 7-10 using 1.0N NaOH. Compound (1) was dissolved in the buffer solution. The solution contained 0.06% Compound (1), 15% povidone, and 100 mM sodium phosphate. The solution was added to lactose/Avicel in a high shear granulator for granulation. If needed, purified water was added to bring the granulation to an appropriate end-point. About 200 g of solution was used per 1 kg of granulation. The wet granules were screened and dried in a fluid bed dryer to a LOD of ˜1%. The dry granules were then milled through a 30-mesh screen. The granulation which contained 0.012% Compound (1) was blended with Pharmatose DCL15 and Avicel PH102 and the blend was lubricated using magnesium stearate. The lubricated blend was compressed into ¼″ round tablets, with a tablet weight of 120 mg, and target hardness of 6-7 kP. The formulation is shown in Table 4:

TABLE 4

| | Unit Formula | |
|---|---|---|
| Ingredients | (mg/tablet) | |
| Compound (1) Granulation | 84.0 | |
| Compound (1) drug substance | | 0.0098 |
| Povidone (Plasdone K29/32) | | 2.44 |
| Sodium Phosphate Monobasic | | 0.22 |
| Water* | | QS |
| Sodium Hydroxide** | | QS |
| Lactose Monohydrate (Lactose Impalpable #312) | | 73.2 |
| Microcrystalline Cellulose (Avicel PH 101) | | 8.13 |
| Lactose Monohydrate (Pharmatose DCL 15) | 8.2 | |
| Microcrystalline Cellulose (Avicel PH 102) | 26.6 | |
| Magnesium Stearate | 1.20 | |
| Total | 120.0 | |

*Water is removed during the drying process
**A 1.0N sodium hydroxide solution is prepared for pH adjustment

The tablets were tested for content uniformity. The results are presented in Table 5.

TABLE 5
Content Uniformity of Tablets containing 10 μg Compound
1 Manufactured by High Shear Granulation Process

| Sample | Content (μg/tablet) |
|---|---|
| 1 | 10.24 |
| 2 | 10.70 |
| 3 | 10.73 |
| 4 | 10.31 |
| 5 | 10.60 |
| 6 | 10.50 |
| 7 | 10.43 |
| 8 | 10.07 |
| 9 | 10.44 |
| 10 | 10.45 |
| Average | 10.45 |
| % RSD | 2.0 |
| Maximum | 10.73 |
| Minimum | 10.07 |

The data in Table 5 indicates that the preferred content uniformity (<3% RSD) was achieved by using the high shear granulation process.

### EXAMPLE 4

### Formulation of Tablets Containing 1 μg/0.1 μg of Compound 1 Tablets by High Shear Granulation Process

The formulation is shown in Table 6 below:

TABLE 6

| | |
|---|---|
| | Unit Formula |
| Ingredients | (mg/tablet) |

| | | |
|---|---|---|
| Compound 1 Granulation | 84.0 | |
| Compound 1 drug substance | | 0.0001-0.001 |
| Sodium Citrate, Dihydrate | | 0.198 |
| Citric Acid, Monohydrate | | 0.0353 |
| Povidone (Plasdone K29/32) | | 2.52 |
| Water* | | QS |
| Sodium Hydroxide** | | QS |
| Lactose Monohydrtate | | 73.1 |
| (Lactose Impalpable #312) | | |
| Microcrystalline Cellulose | | 8.12 |
| (Avicel PH 101) | | |
| Microcrystalline Cellulose | 31.2 | |
| (Avicel PH 102) | | |
| Croscarmellose Sodium (Ac-Di-Sol) | 3.60 | |
| Magnesium Stearate | 1.20 | |
| Tablet Core | 120.0 | |
| Opadry White OY-S-9603 | 3.60 | |
| Water*** | QS | |
| Total | 123.6 | |

*Water was removed during the drying process.
**A 1.0 N sodium hydroxide solution was prepared for pH adjustment.
***Water was removed as part of the coating process.

The manufacturing process can be divided into four steps and is described as follows.

Preparation of Compound 1 Granulation Solution

1) For 1 kg batch size of granulation, 200 g of Compound 1 solution was prepared. The Compound 1 granulation solution contained 50 mM sodium citrate buffer at pH 6.5 and 15% povidone. Drug concentration was dependent on the dose: 0.0006% for 0.1 μg dose and 0.006% for 1 μg dose tablets. The procedure is described below.
2) A solution of 1.0 N sodium hydroxide was prepared. Not less than 50 mL of 1.0N NaOH was prepared for 1 kg of Compound 1 granulation solution.
3) 750 g of purified water was drawn per 1 kg of granulation solution. Under an agitator, sodium citrate was added and dissolved in the purified water.
4) Povidone was dissolved in the sodium citrate solution.
5) Compound 1 drug substance was added to the above solution and slowly added 1.0N NaOH solution under agitation until the drug was completely dissolved. Recommended quantity of 1.0N NaOH for this step was 15-20 g per 1 kg of granulation solution. The solution was protected from light.
6) Citric acid was added to the above solution and the pH was adjusted to 6.5 with the 1.0 N NaOH solution. The solution was brought to the target weight with purified water.
   High Shear Granulation
1) Lactose and microcrystalline cellulose was charged into a high shear granulator and blended until uniform.
2) The Compound 1 granulation solution was slowly poured into the granulator and mixed until uniform.
3) Additional purified water was added to a desired endpoint. About 50 g of additional water was recommended per 1 kg batch size of granulation.
4) The wet granulates were screened.
5) The screened wet granulates were dried in a fluid bed dryer to a LOD of <2%. The inlet temperature was set at about 60-70° C.
6) The dry granules were milled through a 20-30 mesh screen.
   Blending and Tabletting
1) Microcrystalline cellulose and croscarmellose sodium were screened through a suitable screen (20 mesh) and charged into a blender.
2) The Compound 1 granulation was added into the blender and blended until uniform.
3) Magnesium stearate was screened through a suitable screen (40 mesh) and charged into the blender and blended.
4) The blend was compressed on a rotary tablet press with a target weight of 120 mg and target hardness of 6-7 kP.
   Film Coating
1) A suspension of 12% Opadry White OY-S-9603 in purified water was prepared. About 400 g of suspension was prepared per 1 kg of Compound 1 tablets.
2) The Compound 1 tablet cores were coated in a coating pan to a weight gain of approximately 3%.

Batches of Compound (1) 1 μg and 0.1 μg tablets were manufactured by the process of the present invention. The batch of Compound 11 μg tablet was coated with Opadry White OY-S-9603 while the batch of Compound 10.1 μg tablet was not coated. Table 7 lists the content uniformity results.

TABLE 7
Content Uniformity of Compound 1 Table
1 μg/0.1 μg Tablets

| | Compound 1 | Compound 1 |
|---|---|---|
| Sample | 1 μg Tablets | 0.11 μg Tablets |
| 1 | 0.973 | 0.108 |
| 2 | 0.997 | 0.105 |
| 3 | 0.986 | 0.109 |
| 4 | 0.951 | 0.108 |
| 5 | 0.973 | 0.106 |
| 6 | 0.997 | 0.109 |
| 7 | 0.984 | 0.110 |
| 8 | 1.008 | 0.106 |
| 9 | 0.963 | 0.109 |
| 10 | 0.946 | 0.108 |
| Average | 0.98 | 0.108 |
| % RSD | 2.1 | 1.5 |
| Maximum | 1.008 | 0.110 |
| Minimum | 0.946 | 0.105 |

The data in Table 7 indicates that the content uniformity is excellent for 1 μg tablets and even 0.1 μg tablets (<3% RSD). These batches demonstrate that the process of the present invention was suitable for formulating ultra low dose pharmaceutical products at doses of <1 mcg (0.001% by weight) and even nano dose units at doses of equal to or less than 0.1 mcg or 0.0001% by weight. The formulation described in this Example has also been used to prepare tablets containing 1 μg up to 20 μg.

What is claimed is:

1. A method for preparing dosage forms comprising low dose pharmaceutically active substances which comprises admixing carrier particles with a solution comprising the pharmaceutically active substance together with a binder therefor.

2. A method according to claim 1 wherein the dose of pharmaceutically active substance is less than 100 μg.

3. A method according to claim 2 wherein the dose of pharmaceutically active substance is less than 20 μg.

4. A method according to claim 3 wherein the dose of pharmaceutically active substance is less than 1 μg.

5. A method according to claim 1 wherein the ratio of solution comprising drug and binder: carrier is 5-50:100.

6. A method according to claim 5 wherein the ratio of solution comprising drug and binder: carrier is 15-35:100.

7. A method according to claim 6 wherein the ratio of solution comprising drug and binder: carrier is 20-30:100.

8. A method according to claim 1 wherein the dosage form has a desired content uniformity of <7.5% RSD.

9. A method according to claim 8 wherein the dosage form has a desired content uniformity of <6% RSD.

10. A method according to claim 9 wherein the dosage form has a desired content uniformity of <3% RSD.

11. A method according to claim 1 wherein the dosage form is a solid dosage form.

12. A method according to claim 1 wherein the mixing step is carried out in a High Shear Mixer.

13. A method according to claim 1 wherein the mixture is formulated into unit dosage presentations.

14. A pharmaceutical composition comprising a drug obtainable by the method of claim 1.

15. A method according to claim 1 wherein the pharmaceutically active substance is Compound (1) (2-methyl-2-[4-{[(4-methyl-2-[4-trifluoromethylphenyl]-thiazol-5-ylcarbonyl)amino]methyl}phenoxy]propionic acid 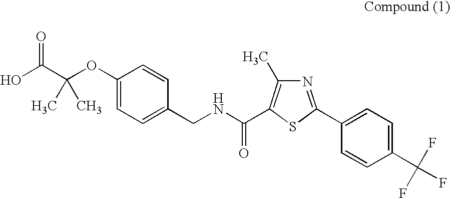
or a pharmaceutically acceptable salt, solvate or physiologically functional derivative thereof.

16. A pharmaceutical composition comprising 1-100 μg of Compound (1) or pharmaceutically acceptable salts, solvates and hydrolysable esters thereof together with a pharmaceutically acceptable carrier.

17. A pharmaceutical composition comprising less than 20 μg of Compound (1) or pharmaceutically acceptable salts, solvates and physiologically functional derivatives thereof together with a pharmaceutically acceptable carrier.

18. A pharmaceutical composition comprising 1-18 μg of Compound (1) or pharmaceutically acceptable salts, solvates and physiologically functional derivatives thereof together with a pharmaceutically acceptable carrier.

19. A pharmaceutical composition comprising 1-10 μg of Compound (1) or pharmaceutically acceptable salts, solvates and physiologically functional derivatives thereof together with a pharmaceutically acceptable carrier.

20. A pharmaceutical composition according to claim 16 wherein Compound (1) or pharmaceutically acceptable salts, solvates or physiologically function derivatives thereof comprises form 2, form 6 and mixtures thereof.

21. A method of treatment of a human PPAR mediated disease or condition comprising administration to a subject a daily dose of 1-100 μg Compound (1) or pharmaceutically acceptable salts, solvates and physiologically functional derivatives thereof.

22. A method according to claim 21 wherein the daily dose of Compound (1)) or pharmaceutically acceptable salts, solvates and physiologically functional derivatives thereof is less than 20 μg.

23. A method according to claim 22 wherein the daily dose of Compound (1)) or pharmaceutically acceptable salts, solvates and physiologically functional derivatives thereof is 1-18 μg.

24. A method according to claim 23 wherein the daily dose of Compound (1)) or pharmaceutically acceptable salts, solvates and physiologically functional derivatives thereof is 1-10 μg.

25. A method according to claim 21 wherein where Compound (1) comprises form 2, form 6 or mixtures thereof.

26-30. (canceled)

31. A method according to claim 21 wherein the Human PPAR mediated diseases or conditions include dyslipidemia including associated diabetic dyslipidemia and mixed dyslipidemia, syndrome X (as defined in this application this embraces metabolic syndrome), heart failure, hypercholesteremia, cardiovascular disease including atherosclerosis, arteriosclerosis, and hypertriglyceridemia, type II diabetes mellitus, type I diabetes, insulin resistance, hyperlipidemia, inflammation, epithelial hyperproliferative diseases including eczema and psoriasis and conditions associated with the lung and gut and regulation of appetite and food intake in subjects suffering from disorders such as obesity, anorexia bulimia, and anorexia nervosa, cancer, Alzheimers disease or other cognitive disorders.
